(19) United States
(12) Patent Application Publication
(10) Pub. No.: US 2012/0087997 A1
(43) Pub. Date: Apr. 12, 2012

(54) EDIBLE PRODUCTS WITH THERMOSTATIC AND COGNITIVE EFFECTS

(75) Inventors: Ascher Shmulewitz, Tel-Aviv (IL); Omri Simon Shmulewitz, Tel-Aviv (IL)

(73) Assignee: Innosense Ltd., Bnei-Brak (IL)

(21) Appl. No.: 13/378,366

(22) PCT Filed: Jun. 8, 2010
(86) PCT No.: PCT/IL10/00453

§ 371 Date: Dec. 15, 2011

Related U.S. Application Data
(60) Provisional application No. 61/218,096, filed on Jun. 18, 2009.

(51) Int. Cl. A61K 31/445; A61P 25/00; A61K 36/67; A61P 3/00; A61K 31/522; A61K 36/33

(52) U.S. Cl. 424/734; 514/315; 514/263.34; 424/767

(57) ABSTRACT

An edible product for normalizing blood sugar levels is provided. The product includes a mulberry extract having a (w/w) flavonoids concentration of at least 10%, a 1-deoxynojirimycin (DNJ) concentration of about 2% and a GABA concentration of about 1% of the total amino acid content of said mulberry extract.

### FIELD AND BACKGROUND OF THE INVENTION

The present invention relates to an edible product containing mulberry extract and to methods of using same for stabilizing a metabolic activity of an individual and improving cognitive functions, as well as mood.

In recent years, consumption of dietary, energy and health products containing extracts or compounds of natural origin as active ingredients has become more and more widespread. Consumption of such products is largely driven by lifestyle and consumer appeal and as such, manufacturers often associate “good-for-you” and “high energy” with such products.

In recent years these products are increasingly marketed in the form of drinks. Such drinks are advertised as activity drinks, energy drinks or nutrient enriched drinks which claim to replenish loss of carbohydrates, electrolytes, vitamins, minerals, amino acids, and other important nutrients or to provide an energy boost to active individuals.

There are numerous ‘health’ drinks, boasting a wide variety of claims, from rehydrating your body to boosting your alertness, from stimulating your metabolism to helping to combat stress and promoting an active life. However, such claims have yet to be substantiated scientifically.

Many popular “energy” drinks are loaded with caffeine or other stimulants and large amounts of sugar which is oftentimes disguised as crystalline fructose or high fructose corn syrup. Energy drinks provide a brief energy boost but once the effect wears off, energy levels drop, leaving the individual even more fatigued then prior to consumption of the energy drink.

These drinks are oftentimes labeled as “all natural” noting that the active ingredients are derived from natural extracts; however, claims on the health benefits afforded by the natural extracts contained in such drinks are questionable due to the fact that they provide a sub clinical dose of active ingredients.

Mulberry (Morus) is a genus of 10-16 species of deciduous trees native to warm temperate and subtropical regions of Asia, Africa, Europe, and the Americas, with the majority of the species native to Asia. Mulberry has been used in Chinese medicine since 659 AD. The Chinese Pharmacopoeia lists the leaves, root bark, branches, and fruit as ingredients in medicinal preparations, but other parts, including the sap and wood ash, are also widely used. Mulberry plant extracts have been used in treatment of diseases like diabetes mellitus, arthrosclerosis, hyperlipidemia, hypertension etc.

At present, the sole use of mulberries in modern medicine is for the preparation of syrup, employed to flavor or color medication. The fact that Mulberry is a rich source of natural isoprenoid substituted phenolic compounds including flavonoids, has motivated researchers to look at the affects of mulberry extracts on human health. For example, Japanese researchers reported that a new mulberry powder, with an optimized content of 1-deoxynojirimycin (DNJ) may be used as a dietary supplement to help prevent diabetes. DNJ has been shown to inhibit the action of the glucosidase enzyme that controls the digestion of carbohydrates [Journal of Medicinal Plants Research Vol. 2(10), pp. 271-278, October, 2008].

While reducing the present invention to practice, the present inventors have formulated an edible product that includes mulberry leaf extract and additional ingredients functioning in synergy to provide health benefits and in particular to stabilize the metabolic activity of an individual.

### SUMMARY OF THE INVENTION

According to one aspect of the present invention there is provided an edible product comprising a mulberry extract having a (w/w) flavonoids concentration of at least 10%, a 1-deoxynojirimycin (DNJ) concentration of about 2% and a GABA concentration of about 1% of the total amino acid content of the mulberry extract.

According to further features in preferred embodiments of the invention described below, the product further comprises at least one ingredient selected from the group consisting of green tea extract, prickly pear extract, black pepper extract, ginseng extract, ginkgo biloba extract, sesame extract, olive extract, sodium, magnesium, potassium, zinc, and a vitamin B complex.

According to still further features in the described preferred embodiments the mulberry is white mulberry.

According to still further features in the described preferred embodiments the mulberry extract is from mulberry leaves.

According to still further features in the described preferred embodiments the mulberry extract includes a flavonoids concentration of at least 20%.

According to another aspect of the present invention there is provided a method of stabilizing metabolic activity comprising administering to a subject in need an edible product comprising a mulberry extract having a (w/w) flavonoids concentration of at least 10%, a 1-deoxynojirimycin (DNJ) concentration of about 2% and a GABA concentration of about 1% of the total amino acid content of the mulberry extract.

According to still further features in the described preferred embodiments the subject in need is suffering from affects of excessive alcohol consumption.

According to still further features in the described preferred embodiments the subject in need is suffering from affects of excessive sugar, caffeine or other stimulant consumption.

According to still further features in the described preferred embodiments the subject in need is suffering from stress, nervousness, fatigue, disorientation, nausea, irritated gastrointestinal function, upset mood, loss of cognitive functions, hyperactivity, attention problems, insomnia or restlessness or lack of sex drive.

According to still further features in the described preferred embodiments the subject in need is suffering from affects of prolonged flight environment.

According to another aspect of the present invention there is provided an article of manufacturing comprising an edible product including a water soluble mulberry extract having a (w/w) flavonoids concentration of at least 10%, a 1-deoxynojirimycin (DNJ) concentration of about 2% and a GABA concentration of about 1% of the total amino acid content of the mulberry extract and packaging material identifying the edible product in treatment of the affects of excessive alcohol consumption.

The present invention successfully addresses the shortcomings of the presently known configurations by providing an edible product useful in stabilizing the metabolic activity of an individual and in particular the blood sugar level of the individual.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a prior art graph showing the post meal sugar benefit of Glucocil, a mulberry leaf-based dietary supplement.

### DESCRIPTION OF THE PREFERRED EMBODIMENTS

The present invention is of an edible product which can be used to stabilize the metabolic activity of an individual and thus counteract or balance the deleterious effects of short term stimulants or depressants as such as caffeine or alcohol.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description, Examples or illustrated in the drawings. The invention is capable of other embodiments or of being practiced or carried out in various ways. Also, it is to be understood that the phraseology and terminology employed herein is for the purpose of description and should not be regarded as limiting.

The stresses of living in a modern western society combined with excessive consumption of stimulants or depressants leads to short or long term metabolic imbalances in many individuals. As is further described hereinunder, the present inventors formulated an edible product which includes a mulberry (Morus alba) extract in combination with additional herbal extracts, short-term/fast-burning carbohydrates (e.g. sucrose) and long-term/slow-burning carbohydrates (e.g. isomaltulose) which is capable of stabilizing metabolic activity and thus can provide health benefits to individuals having short or long term metabolic imbalances.

Thus, according to one aspect of the present invention there is provided an edible product which can be used to stabilize the metabolic activity of a subject such as a human. As such, the edible product of the present invention can be used, for example, to balance the physiological effects of stimulants or depressants. This balancing activity of the edible product of the present invention is termed herein, as “thermostatic activity”. As opposed to thermogenics and short-term stimulants such as caffeine, the thermostatic edible product of the present invention is formulated in order to maintain an optimal and constant metabolic state over time.

While caffeine and sugar consumption boost energy levels, these benefits are short-lived and cause rapid increases and decreases in glucose blood-sugar levels. This is commonly referred to as the energy “crash”. Similarly, albeit a depressant, alcohol creates an illusion of boosted energy and morale, in particularly when consumed alongside sugar and caffeine. This, however, is also followed by a sudden drop in physiological and mental states.

The present invention can be used in preparation for, during and/or following activities which produce metabolic imbalance and/or its associated effects, such as stress, fatigue, dehydration, hyper/hypoglycemia, nausea, hangover, disorientation, etc. It is an alternative to consumption of caffeine and other stimulants—providing a natural, sustained peak without the imbalance that follows. It can be used for enhancing and maintaining mood and physical performance throughout demanding daily routine, and at critical points of transition and/or recovery such as early morning work/study hours, following meals (“the 3 o'clock down”), during or after recreational activity, and at any point when the prevention or reduction of physiological effect caused by consumption of alcohol, caffeine and sugar is needed.

The ability of the present composition to balance blood sugar levels can also be used to improve cognitive functions and control Attention Deficit and Hyperactivity Disorder (ADHD) in adults and children. It has been suggested that a side effect of many AMID medications is a change in blood sugar and glucose metabolism (see, www. well sphere.com/add-adhd-article/adhd-methylphenidate-and-blood-sugar-levels/663143). Ingestion of the present composition prior to, concomitant with, or following administration of ADHD medication can balance blood sugar levels in individuals medicated for AMID and other attention deficit disorders.

As used herein, the phrase “edible product” refers to any product that includes active ingredients that are ingested by a human subject. Such a product need not be swallowed in order for the active ingredients to be consumed. For example, an edible product in the form of a chewing gum includes a base (a gum base, e.g. latex or wax) which releases the active ingredients and thus the product itself need not be swallowed for the active ingredients to be consumed.

The edible product can be formulated in any form suitable for ingestion; examples include, but are not limited to, a carbonated or non-carbonated drink, an alcoholic beverage, a refreshing drink, an herbal tea, a nutritional supplement, a syrup, a juice, a functional mineral water, a liquid carrier, a dried food product, a hard or soft candy, a cheese, a capsule, a powder, a tablet, a pill, an ice cream, a baked good, a snack, a drink mix, an energy or cereal bar, a chewing gum, a paste, a soy-processed food, a mousse, a jelly, a yogurt, a chocolate, etc.

The edible product of the present invention includes a water soluble mulberry (Morus Alba) leaf extract having a (w/w) flavonoids concentration of at least 10% (preferably 20% or more), a 1-deoxynojirimycin (DNJ) concentration of about 2% and a GABA concentration of about 1% of the total amino acid content of said mulberry extract.

The extract of the present invention is a combination of White Mulberry Fruit Powder Extract prepared by Naturex, with 10% flavonoids content, obtained from mulberry fruits at a ratio of 10/1; and a White Mulberry Leaf Powder Extract prepared by Naturalinbio, with 20% flavonoids content and 2% DNJ.

Leaf extraction is preferably effected using fresh and green undamaged leafs which are dried and ground to a uniform particle size. The ground leaves are then mixed with Grain Alcohol and the resulting mash is blended and subjected to series of solvent extraction steps with a solvent recovery process for re-use. The leaf extract is dried and the resulting crystals are further dehydrated using a high speed centrifugal mixer. Final moisture extraction is effected via a drying oven.

Mulberry is renowned as the primary food source for silkworms, and as such has been widely cultivated in its native China for 5000 years. There are four main varieties: black, red, white and mountain/wild; which over centuries have diversified to hundreds of varieties in almost any altitudes and climates in the world.

White Mulberry (Morus Alba) is the species that has been used exclusively in Chinese medicine as a “cooling” herb to help detoxify and cool-down an overheated body. It is considered an ant febrile (anti-fever) and antiphlogistic (anti-inflammatory) agent. Thus it is used to clear the liver, and treat influenza, bronchitis, headaches, dizziness, coughs, and a swollen and sore throat.

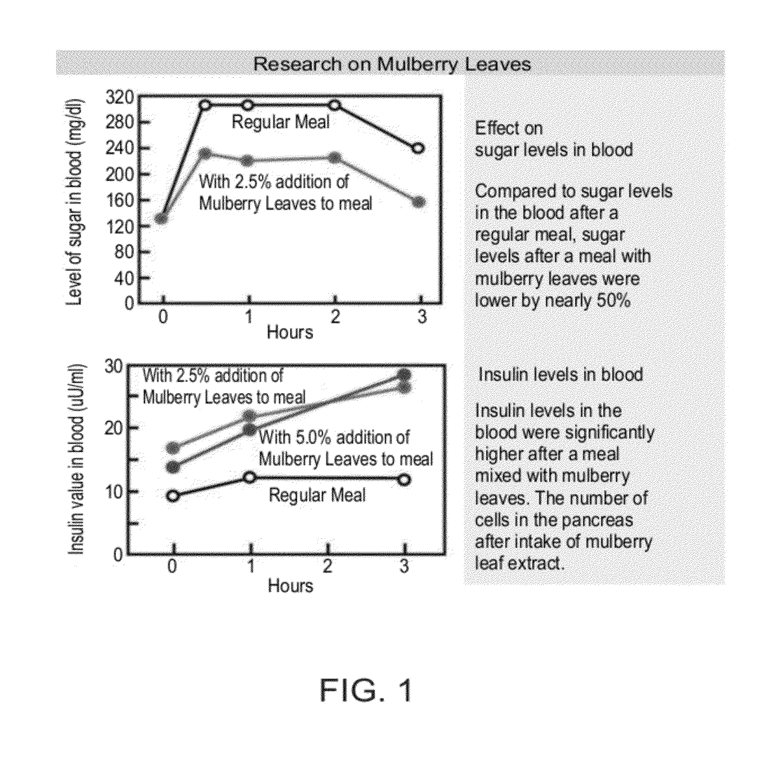

Mulberry is a source of alkaloidal sugar-mimic glycosidase inhibitors such as 1-Deoxynojimycin (DNJ). DNJ has been found effective in controlling and reducing carbohydrate absorption and decreasing (post-meal) high blood-sugar levels. This has been utilized in the commercial diabetic herbal supplement Glucocil which has been shown to have an affect on blood glucose levels (see FIG. 1).

Mulberry is rich in Flavonoids (e.g. quercetin) which are powerful antioxidants with unique metabolic properties. Hypoglycemic and antioxidant potency of several phenolic compounds (Flavonoids, stilbenes and 2-arylbenzofurans) has been reported. Quercetin has been shown to provide sustained energy by mimicking effects of exercise and enhancing brain and muscle mitochondrial biogenesis and exercise tolerance. Mulberry Leaf extract has been shown to stimulate 5′-AMP-activated protein kinase (AMPK)—a metabolic “fuel gauge” at cellular level—in isolated rat skeletal muscle. AMPK activity is increased during exercise in an intensity and glycogen dependant manner. Thus, mulberry extracts also posses an “endurance energy” property.

Nutritional extracts and teas based on mulberry are sold with claims to promote healthy digestive system, lower high blood pressure and cholesterol, control body weight, support healthy liver function and serve as a detoxifying agent. Some also cite, among others: calming the nerves, enhancing immunity, strengthening eyesight, balancing internal secretions, treating constipation and promoting the metabolism of alcohol.

Mulberry leaves contain a rich essential amino acid profile, and is a natural source of the amino-acid GABA, the major inhibitory neurotransmitter in the brain.

The mulberry extract is used by the present invention as a functional agent for generating sustained state of improved metabolism and normalization of blood-sugar, restoring the body to its “optimal zone”, preventing the ‘highs’ and ‘lows’ that follow (hyper or hypoglycemia); cooling an overheated body and quickening removal of toxins and free radicals; enabling “endurance energy” (as mentioned above); and improving mood and relief of stress.

The edible product of the present invention also includes additional synergically-acting herbal extracts, long-acting and short-acting carbohydrates, and minerals to provide a balanced and a stabilizing effect on metabolism over time.

Chinese medicine utilizes a group of several herbs that have a similar end effect, but with different active components and different mechanisms. For example, a formula which includes Morus fruit, ho-shou-wu, rehmannia, and ligustrum may nourish blood, alleviate constipation, and have antioxidant and anti-aging effects, but each herb contributes a different group of active components: anthocyanins, anthoquinones, iridoid glycosides, and oleanolic acid, respectively. Together, these may reduce adverse oxidative reactions, enhance circulation, alleviate inflammatory processes that yield systemic consequences, and improve the digestion and absorption of nutrients while enhancing the elimination of waste products of metabolism.

Similarly, additional extracts that produce a synergic effect to that of mulberry extract can also be utilized. Use of several herbal extracts in conjunction can provide a more beneficial functionality that may surpass that of each extract alone, or that of the active compounds found in the extracts themselves.

Such extracts should contribute additional phytonutrients and compounds that function in conjunction to supplement the “thermostatic” activity of the mulberry extract. These compounds should function in detoxifying and rehydrating or as antioxidants, anti-inflammatory agents, metabolic endurance (blood-sugar control) agents or mood-enhancers/stress-relievers. Examples of herbal extracts that can be used in the present invention are listed below along with the rational for using such extracts in combination with the mulberry extract.

Two preferred extracts are Green Tea Extract and India Fig (Prickly Pear) Fruit Extract; both can be utilized at ratio of about 1 to 7 with respect to the mulberry extract (w/w). For example, in a drink product, 1.5 g mulberry/liter can be combined with 0.2 g green tea per liter and/or 0.2 g Indian fig per liter.

Green tea—Camellia sinensis (“green tea”) is possibly the most ancient and widespread functional food or medicine in the world. It is rich in catechin polyphenols, particularly epigallocatechin gallate (EGCG). EGCG is a powerful anti-oxidant, it inhibits growth of cancer cells and also kills cancer cells without harming healthy tissue. It has also been effective in lowering LDL cholesterol levels, and inhibiting the abnormal formation of blood clots. Green tea can increase metabolic rate, and endurance during exercise. It is proposed herein that a synergistic interaction between the catechin-polyphenols and caffeine of green tea provides prolonged sympathetic stimulation of thermogenesis.

Green tea is generally considered to assist in fighting cancer, stabilizing diabetes, helping in weight loss, slowing the ageing process, enhancing immunity, fighting bacteria and helping digestion, etc.

L-theanine is a free amino acid found almost exclusively in tea plants. This compound provides the unique physiological effect of tea, seemingly contradictory to the stimulatory effect of caffeine (which is also present in green tea). Theanine has been shown to reduce anxiety, enhance mood, increase alpha waves and alertness and counteract the negative side-effects of caffeine. Furthermore, it has previously been shown that theanine positively effects alcohol-metabolism by increasing alcohol dehydrogenase and aldehyde dehydrogenase activities in the liver.

The present invention can utilize a water-soluble extract of Green Tea which provides between 50-95% polyphenols and 20-90% catechins. Standardized amount of theanine and or EGCG are preferred.

Indian Fig—This plant is a member of the Cactacea (Cactus) family, known as Opuntia ficus indica. Some of the names by which the Opuntia is also known are Barbary Fig, Indian Fig, Nopal or, most commonly, the Prickly Pear. Since pre-historic times, desert natives in North and South America have consumed the fruit and pads of this member of the Cactaceae family. Spanish explorers carried this and other cacti species to Europe over two centuries ago and not long after, the Moors introduced the Opuntia to Africa.

Opuntia ficus indica fruit possess potent antioxidative properties. The cladodes of the plant have been used in traditional medicine in many countries and are rich in mucilage which consist primarily of polysaccharides. This plant includes several compounds that have been shown to provide blood-sugar controlling activity. The hypoglycemic activity of a purified extract from prickly pear cactus was successfully evaluated on diabetic rats in which blood glucose was reduced to normal values by a combined treatment of insulin and Opuntia extract.

Opuntia ficus indica cab be used to alleviate symptoms of alcohol hangover. The severity of the alcohol hangover may be related to inflammation induced by impurities in the alcohol beverage and byproducts of alcohol metabolism. An extract of the Opuntia ficus indica (OFI) plant diminishes the inflammatory response to stressful stimuli, proving a moderate effect on reducing hangover symptoms. More specifically, it was shown that the unique environmental defense mechanisms of the cactus were centered on its ability to rapidly induce the synthesis of protective heat shock proteins—compounds that play a natural protection and recovery role against physical stresses and their negative side-effects, such as in alcohol consumption.

Example of OFI extract that may be utilized in the current invention is Tex-OE, a Prickly Pear fruit extract, produced by Nutrimak.

Thus, an edible product based on mulberry extract and including Green Tea and India Fig extracts would provide numerous health benefits.

Bioperine—standardized black pepper extract, which is a bioavailability enhancing thermonutrient, a metabolic enhancer at cellular level (thermogenesis) providing a more efficient mode of nutrient transportation into the blood. Preferably added in small quantity to the present invention in order to assist in boosting beneficial effect of other components (herbal extracts and minerals).

In addition to the above described extracts, the present invention can also include various minerals and/or electrolytes, such as sodium, magnesium and potassium. Such components are essential for re-hydration and for optimal performance during and in-between (replenishing/recovery) strenuous activities as well as following consumption of alcohol and caffeine.

Sodium Citrate and Potassium Citrate—used in the present invention at a ratio of about 2 to 1 for optimal hydration.

Magnesium Chloride—use in the present invention at a ratio of about 1 to 5 to the sodium content. Dehydration occurs during strenuous activity (sweating), and more excessively through diarrhea, vomiting or alcohol consumption. Diabetes and/or elevated blood-sugar levels can also cause significant dehydration. Magnesium and Potassium are essential nutrients for boosted metabolism and are depleted following alcohol intake.

Gamma-Amino Butyric acid (GABA) is an amino acid which acts as a neurotransmitter in the central nervous system. It inhibits nerve transmission in the brain, calming nervous activity. As a supplement it is sold and promoted for these neurotransmitter effects as a natural tranquilizer. It is also touted as increasing Human Growth Hormone levels and is popular among body builders. GABA is found naturally in mulberry leaves, which can be manipulated to provide higher GABA content, and can also be further added to the present product as a supplement. GABA hinders the transmission of nerve impulses from one neuron to another. It has a calming or quieting influence. Caffeine inhibits GABA release, resulting in increased nerve transmission and a stimulatory effect. It has been suggested that GABA positively effects metabolism of alcohol.

Alcohol consumption initially produces feelings of relaxation and cheerfulness, but further consumption can lead to blurred vision and coordination problems. The initial effect may be due to increased permeability of GABA through the Blood Brain Barrier when combined with alcohol. Consuming a mulberry extract that is GABA-enriched and also includes water, minerals, and carbohydrates can be used to prevent or lessen the effects of a hangover, and reduce the variability in the metabolism of ingested foods, including alcohol. Furthermore, variability in metabolism of carbohydrates and alcohol between individuals suggests a benefit to a food supplement enriched with glutamic acid derivatives (e.g. GABA and Theanine).

Isomaltulose is a disaccharide that is commercially produced enzymatically from sucrose. It is a natural constituent of honey and sugar cane and has a very natural sweet taste. It has been used as a sugar in Japan since 1985. It is particularly suitable as a non-cariogenic sucrose replacement and is favorable in products for diabetics and prediabetic dispositions.

Unlike sucrose, isomaltulose is digested much slower, leading to a low glycemic response and a prolonged glucose (energy) supply. In the present invention, utilization of several kinds of carbohydrates with rapid and/or slow catalysis, can further assist in balancing metabolic (“thermostatic”) activity. Specifically, the edible product of the present invention can be formulated so as to provide fast energy release compounds (vital particularly following hypoglycemia, caused by various unbalancing metabolic factors, as described above), as well as slow energy release compounds—thus providing an “endurance-energy” functionality.

The present invention can utilize both sucrose and palatinose (isomaltulose) as a source of simple carbohydrates.

Furthermore, the composition of the invention can contain additional ingredients, such as medicinal or other herb, amino acid, vitamin, minerals as well as inert or functional ingredients typically used in edible products. Active ingredients can supplement functionality described herein (such as that attributed to Flavonoids or GABA), or provide synergic activity. There is no specific limitation regarding described materials. Herbal extracts can include water soluble or organic soluble compound.

As is mentioned hereinabove, the edible product of the present invention can be formulated in any form suitable for human consumption. One preferred form is a water-based carbonated drink which is easily consumed and facilitates rapid absorption of the active ingredients. Such a drink can be formulated as follows:

1. Base:
Carbonated Water—1000 g/Litre
Citric Acid—1.6 g/Litre
Bitter Lemon—4 g/Litre

2. Herbal Extract:
Mulberry Leaf Extract—1.5 g/Litre.
Green Tea Extract—0.2 g/Litre
Indian Fig Fruit Extract—0.2 g/Litre
Bioperine (Black Pepper Extract)—0.005 g/Litre

3. Minerals:
Sodium Citrate—0.5 g/Litre
Potassium Citrate—0.3 g/Litre
Magnesium Chloride—0.15 g/Litre

4. Carbohydrates
Sucrose—65-85 g/Litre
Isomaltulose—5-20 g/Litre

5. Natural Flavours.

Serving size for described carbonated drink is 250 ml. Container label information:
Ingredients: Water, Sucrose, CO2, Lemon Base, Citric Acid, Isomaltulose, Herbal Extracts (Morus Alba, Green Tea, Opuntia Ficus-Indica, Black Pepper), Natural Flavours, Natural Food Colouring Beta Carotene, Quinine, Minerals (Sodium Citrate, Potassium Citrate, Magnesium Chloride).
Nutritional Value (/100 ml):
Calories 37
Carbohydrates 9.5 g
Fat 0 g
Protein 0.1 g
Sodium 50 mg
Magnesium 15 mg
Potassium 30 mg.

Thus, the present invention provides an edible product with “thermostatic activity”, which functions to enhance and stabilize metabolism, mood and cognitive functions as well as assist in detoxifying and rehydrating the body.

The edible product of the present invention provides the following functionality:
(i) “Retunes” the body to its optimal metabolic zone;
(ii) Provides endurance energy and maintains a blood-sugar balance;
(iii) Quickens removal of toxins and counteracts body overheating;
(iv) Scavenges free radicals, thereby relieving oxidative stress, and acting as an anti-inflammatory;
(v) Provides hydration and replenishing of nutrients during and following strenuous activity including alcohol consumption, heating, sweating, excessive urination etc; and
(vi) Enhances mood, focuses and calms the mind while improving coordination and sex drive.

The composition of the present invention can be used by any individual, it will be appreciated however, that individuals with reduced cognitive functions, attention problems and/or episodes of hyperactivity (e.g. individuals having ADHD) would greatly benefit from the metabolism-balancing feature of the composition of the present invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### Examples

Reference is now made to the following examples, which together with the above descriptions, illustrate the invention in a non limiting fashion.

### Example 1

### Alcohol Consumption

As described above, the present invention can be used to prevent or correct a metabolic imbalance and alleviate its negative side-effects, and as such can be used both as an alternative to stimulant consumption and as a recovery facilitator.

One particular use for the present invention is in improvement of alcohol metabolism and protection or minimization of symptoms associated with excessive alcohol consumption. Alcohol consumption can lead to physically and emotionally stress. The diuretic effects of alcohol lead to dehydration and loss of salts and vital electrolytes; these unwanted effects can be further increased by intense physical activity often associated with alcohol consumption (i.e dancing). It has been suggested that excessive uptake of alcohol leads to production of free radicals, a decrease of GSH level, and an increase in the amount of lipid peroxide (LPO) in liver. It has also been shown that the severity of the alcohol hangover may be related to inflammation induced by impurities in the alcohol beverage and byproducts of alcohol metabolism. Furthermore, alcohol intake, especially when accompanied by intake of sugar and caffeine rich beverages, leads to hypoglycemia. In this case, synergic action of present invention as described above can function to increase alcohol dehydrogenase and aldehyde dehydrogenase activities in the liver; normalize blood-sugar; scavenge free radicals; improve lipid peroxidation; diminish inflammatory response to stressful stimuli; improve hydration and provide replenishing of lost electrolytes and relieve nervousness and stress (and additional symptoms caused by ‘wearing off’ of the depressive effect of alcohol).

### Example 2

### High Altitude Situations

An additional use for the present invention is in alleviating the effects of high altitude situations, such as those experienced during prolonged flights. It is under these situations that dehydration and other physically stressing physiological effects are pronounced, and even more accentuated when combined with alcohol consumption. It is known that the impact of alcohol on the body may be 2-3 times more potent when flying. Following these situations, many individuals may find themselves suffering from the negative effects of dehydration, alongside general fatigue, stress and nervousness. While crossing of time zones is the factor generally attributed to so-called “jet lag”, dry atmosphere, cabin pressure, stale air aboard aircraft and lack of exercise all contribute to creating this unwanted effect. In this respect, the present invention can be used in preparation, during and following flights to deter or lessen the negative effects of flying.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An edible product comprising a mulberry extract having a (w/w) flavonoids concentration of at least 10%, a 1-deoxynojirimycin (DNJ) concentration of about 2% and a GABA concentration of about 1% of the total amino acid content of said mulberry extract.

2. The edible product of claim 1, wherein the product further comprises at least one ingredient selected from the group consisting of green tea extract, prickly pear extract, black pepper extract, ginseng extract, ginkgo biloba extract, sesame extract, olive extract, sodium, magnesium, potassium, zinc, and a vitamin B complex.

3. The edible product of claim 1, wherein said mulberry is white mulberry.

4. The edible product of claim 1, wherein said mulberry extract is from mulberry leaves.

5. The edible product of claim 1, wherein said mulberry extract includes a flavonoids concentration of at least 20%.

6. A method of stabilizing metabolic activity comprising administering to a subject in need an edible product comprising a mulberry extract having a (w/w) flavonoids concentration of at least 10%, a 1-deoxynojirimycin (DNJ) concentration of about 2% and a GABA concentration of about 1% of the total amino acid content of said mulberry extract.

7. The method of claim 6, wherein said subject in need is suffering from affects of excessive alcohol consumption.

8. The method of claim 6, wherein said subject in need is suffering from affects of excessive sugar, caffeine or other stimulant consumption.

9. The method of claim 6, wherein said subject in need is suffering from stress, nervousness, fatigue, disorientation, nausea, irritated gastrointestinal function, upset mood, loss of cognitive functions, hyperactivity, attention problems, insomnia or restlessness or lack of sex drive.

10. The method of claim 6, wherein said subject in need is suffering from affects of prolonged flight environment.

11. An article of manufacturing comprising an edible product including a water soluble mulberry extract having a (w/w) flavonoids concentration of at least 10%, a 1-deoxynojirimycin (DNJ) concentration of about 2% and a GABA concentration of about 1% of the total amino acid content of said mulberry extract and packaging material identifying said edible product in treatment of the affects of excessive alcohol consumption.
